(19) United States
(12) Patent Application Publication
(10) Pub. No.: US 2011/0086077 A1
(43) Pub. Date: Apr. 14, 2011

(54) SILICONE HYDROGELS FOR TISSUE ADHESIVES AND TISSUE DRESSING APPLICATIONS

(75) Inventors: Keith R. McCrea, Lafayette, CA (US); Robert S. Ward, Lafayette, CA (US); Yuan Tian, Alameda, CA (US)

(73) Assignee: The Polymer Technology Group, Inc., Berkeley, CA (US)

(21) Appl. No.: 12/745,509

(22) PCT Filed: Nov. 21, 2008
(86) PCT No.: PCT/US08/84306

§ 371 Date: Nov. 24, 2010

Related U.S. Application Data
(60) Provisional application No. 60/990,866, filed on Nov. 28, 2007.

(51) Int. Cl. A61L 15/22; A61K 38/39; A61P 17/02

(52) U.S. Cl. 424/402; 424/445; 514/17.2

(57) ABSTRACT

A silicone hydrogel formulation may contains random and/or block copolymers or oligomers or macromers. The silicone copolymer is copolymerized or blended with other polymers or monomers or macromers to obtain final formulation. The silicone hydrogel may contain crosslinking groups to provide a complete or partially crosslinked final structure. The silicone hydrogel formulation may be pre-formed as a film or other structure, or it may be polymerized during application as in the case of an adhesive formulation. A wound dressing comprising a silicone hydrogel formed as a film, either prior to application to a wound or in situ on a wound, which film has gas permeability, moisture permeability, and high water content, wherein said silicone hydrogel is formed from a polymerizable silicone such as a difunctional polydimethylsiloxane methacrylate and crosslinking agents such as N,N-dimethyllacrylamide (DMA), 2-hydroxyethyl methacrylate (HEMA), and trimethylsiloxy silane (TRIS).

### FIELD OF THE INVENTION

The present invention relates to dressings for wound care, wherein the dressings are made from silicone hydrogels. In the present invention, high oxygen permeability contributed by hydrophobic domains enhances healing, while high water content provided by hydrophilic networks allows for a high moisture transmission rate needed for wound drainage. Silicone hydrogel formulations usable in this invention may be random and/or block copolymers/oligomers/macromers. They may be copolymerized/blended with the other polymers/monomers/macromers. The silicone hydrogels may contain crosslinking groups to obtain a completely or partially crosslinked final structure. The silicone hydrogel formulations may be pre-formed as films or other structures, or they may be polymerized during application, such as in the case of spray-on adhesive formulations.

### BACKGROUND OF THE INVENTION

Silicone hydrogels are a unique class of materials that provides high oxygen permeability while maintaining high water content in the bulk. These properties make silicone hydrogels ideal materials for many biomedical device applications. For example, the eye is very sensitive to foreign materials and so materials selection for ophthalmic applications is critical. Carefully formulated silicone hydrogels exhibit superior comfort when used in contact lenses due to its lack of stinging agents, low modulus, lubricious surface, high oxygen permeability, and high water content. Wounds, either superficial or chronic, are also extremely sensitive to foreign materials, although often not felt or noticed by the patients. Many materials currently used in wound care were not optimized to be in contact with injured tissue. Silicone-hydrogels, on the other hand, were developed specifically for contact lenses and were optimized for direct contact with the sensitive cornea. In fact, material requirements for ophthalmic applications can be used as a good indicator for whether a material is truly appropriate for open wounds. For example, gauze or other wound dressings would be extremely uncomfortable if placed in contact with the eye. This begs the question of whether such materials are a good choice for wound dressings. Many of material requirements for ophthalmic applications are also common to wound dressings: they should not absorb/activate proteins, especially coagulation and complement factors, they should be lubricious so they don't re-injure the wound bed by friction/abrasion, they should have high oxygen permeability to aid in wound healing, they should have a high water content and/or water permeability for moisture control, and they should be flexible for comfort. Thus, silicone hydrogels are therefore candidate materials for use in wound care applications.

### Moisture Control of Silicone Hydrogels

It is now generally accepted that a wound bed must remain moist for improved healing. Thus, a material must provide a moist environment while still allowing a high moisture vapor transmission rate (MVTR) to prevent overhydration. The MVTR allows the removal of exudates, while the high water content will prevent the wound surface from desiccating. Studies have shown that a moist wound environment enhances fibroblast proliferation, encourages collagen synthesis, endothelial cell proliferation, and leads to angiogenesis and wound contraction. D. W. Brett, A Review of Moisture-Control Dressings in Wound Care, J. Wound Ostomy Continence Nurs. 2006; 33(65):S3-S8. Hydrocolloid dressings provide a moist wound environment and low MVTR and have been shown to be superior over traditional gauze dressings with a high MVTR and low moisture retention. Moist wounds, provided by hydrocolloids, have also shown to reduce the rate of infection as compared to gauze dressings.

A silicone hydrogel can have a water content (and permeability) that depends on the concentration of hydrophilic moieties, but which may exceed 50 weight %. For example, the silicone hydrogel Galyfilcon A has a water content of 47%. Its relatively high water absorption therefore enables a silicone hydrogel to maintain a moist wound environment.

### Oxygen Permeability of Silicone Hydrogels

Oxygen also plays an important role in healing and the lack of oxygen has been identified as one of the most common causes of wound problems. Bok Y. Lee “The Wound Management Manual”, McGraw-Hill, New York, 2005, p. 44. Oxygen delivered by hemoglobin is important during wound healing, however, damaged tissue can act as a barrier to hemoglobin leading to localized hypoxia at the wound site. Therefore, ambient oxygen from the atmosphere may be the only source of oxygen for the exterior of wounds. Damaged tissue is generally hypoxic due to the large consumption of oxygen by cells. Leukocytes consume oxygen to produce infection fighting oxidants. In addition, fibroblasts and endothelial cells also require oxygen for wound healing. Thus a low oxygen level at the wound site prevents angiogenesis which prevents the deposition of collagen.

To offset the reduction in infection resistance and wound repair capability of hypoxic wounds, a wound dressing should also provide high oxygen permeability. Hydrocolloids, as discussed above provide a moist environment, but are effective barriers to oxygen. Without silicone the oxygen permeability of a hydrogel is related by a power-law relationship to the hydrogel's equilibrium water content.

### SUMMARY OF THE INVENTION

Silicone hydrogels are employed in the present invention to provide both oxygen permeability and high water content. These properties make these materials suitable for many bio-medical devices such as contact lenses and wound dressings. With regards to wound care applications, high oxygen permeability contributed by hydrophobic domains enhances healing, while high water content provided by hydrophilic networks allows for a high moisture transmission rate needed for wound drainage. In addition, biocompatible silicone hydrogels that are free of irritating agents/groups, exhibit a reduced tendancy to absorb or activate proteins, and have a lubricious surface can provide a potentially skin-like environment to promote wound healing. Disclosed herein are unique silicone hydrogel based materials that can be formulated to be either biostable or degradable. These materials are applied either as a spray, liquid, as a dressing, or a combination thereof. For tissue adhesive applications, chemical components can be included in the formulation that will promote strong bonds to tissue and provide strength for cuts or lacerations. These chemical components responsible for adhesion can be degradable to facilitate release from the wound site after healing has occurred. For burn or skin ulcer applications, materials will be applied to cover the entire wound site. Bonding can occur either at the perimeter of the wound or within the wound itself. In addition to high-oxygen permeability and high moisture transmission, antimicrobial functionality can also be added to prevent infection.

Inclusion of silicone in the hydrogel can significantly increase oxygen permeability at comparable water content allowing silicone hydrogels to be prepared with much higher oxygen permeability than conventional hydrogels and hydrocolloids. The combination of high and controllable oxygen permeability and high and controllable moisture content therefore allows silicone hydrogels to be a suitable wound dressing material. Optimization of wound dressings for different types of wounds and different stages of wound healing is also possible via variations in the hydrophilic content and the silicone concentration during synthesis of the silicone hydrogel monomer mixture. This potential to vary monomer composition to obtain different levels of oxygen and water permeability, and the physical-mechanical properties of the resulting wound dressing may be used empirically to tailor the properties of wound dressings to maximize healing rate and minimize scaring, while protecting the wound from infection by external pathogens. The generally high permeability of silicone hydrogels to both hydrophilic and hydrophobic permeants allows the controlled release of drugs at the wound site, and even allows anti-microbials e.g., iodine, silver, antibiotics, growth factors, peptides, proteins, etc. to be applied topically to the air-facing side of the dressing to diffuse through the dressing to the wound. Anti-microbial polysaccharides like heparin may also be incorporated into silicone-hydrogel wound dressing by covalent, e.g., end-point attachment, admixture within the monomer mix and/or topical treatment.

In one embodiment, this invention provides a wound dressing which comprises a silicone hydrogel formed as a film that has gas permeability, moisture permeability, and high water content. This silicone hydrogel is produced by the reaction of a polymerizable silicone such as a difunctional polydimethylsiloxane methacrylate and crosslinking agents such as N,N-dimethylacrylamide (DMA), 2-hydroxyethyl methacrylate (HEMA), and trimethylsiloxy silane (TRIS). For example, the silicone hydrogel formulation is composed of a copolymer of mono- or di-functional polydimethylsiloxane methacrylate, DMA, HEMA, and TRIS, with or without additional crosslinking agents such as EGDMA. In a specific instance of the invention, the polymerizable silicone may be a mono-(dimethacryloxypropoxypropyl)-polydimethylsiloxane crosslinker which has two methacrylate end groups and in which the chain length n of the PDMS segment (repeating unit —(SiO)n-) in the molecule ranges from 1-20.

The wound dressing of this invention may be bandaging that is pre-formed as a film from a composition comprising 15-35 weight-% difunctional PDMS methacrylate, 10-35 weight-% DMA, 10-35 weight-% HEMA, and 15-30 weight-% TRIS. Alternatively, the wound dressing of this invention may be sprayed on a wound as a spray of a composition comprising 15-30 weight-% difunctional PDMS methacrylate, 10-35 weight-% DMA, up to 20 weight-% HEMA, 10-25 weight-% TRIS, and 15-35 weight-% water. In the spray-on embodiment of this invention, the wound dressing composition may include an initiator, a mono- or di-functional polydimethylsiloxane methacrylate, DMA, HEMA, and TRIS, with a spraying solvent. It may be configured so that when the dressing is sprayed onto a wound, polymerization is started by water or air or light or by heat initiation of the radical polymerization.

The wound dressing of the present invention may be formulated without water or with up to 5% water for use with wet wounds. For use with dry wounds, it may be formulated with 40% to 60% water for use with dry wounds.

The spray-on wound dressing embodiment of this invention may include mono- or di-functional polydimethylsiloxane isocyanate, polydimethylsiloxane-copolymer diisocyanate, polyethyleneglycol, Jeffamine, and catalyst. It may be formulated so that it can be sprayed onto a wound or applied to the wound as a paste. Upon application to a wound of this embodiment of the invention, the isocyanate reacts with diol or diamine to form a silicone polyurethaneurea hydrogel. This wound dressing formulation may further contain polyvinylpyrrolidone (PVP) with a molecular weight in the range 100 to 10 million, so that the PVP may flow to the dressing surface upon hydration to form a lubricious layer. Alternatively, this wound dressing formulation may further contain polyethyleneoxide (PEO) with a molecular weight of 100 to 10 million, so that the PEO flows to the dressing surface upon hydration to form a lubricious layer. In both of these latter embodiments, the amount, structure and/or molecular weight of the PVP or PEO may be controlled to allow for a precalculated removal by washing or absorption, so that the moisture content and oxygen permeability is thereby programmed into the dressing to meet the needs of staged/phased wound healing.

The silicone hydrogel film-type wound dressing of this invention may be placed in contact with a wound and held in place by tape or other secondary support methods. Fabric- or polymer-reinforced silicone hydrogel film-type wound dressings in accordance with this invention may be placed in contact with a wound and held in place by tape or other secondary support methods, wherein the reinforcement layer is optionally a moisture barrier to prevent dehydration of the silicone hydrogel. When a silicone hydrogel film-type wound dressing of this invention is placed in contact with a wound and is held in place by an adhesive around the wound site, the adhesive may be either placed topically prior to dressing the wound, or incorporated into the film during manufacture. The reinforcement layer is optionally a moisture barrier to prevent dehydration of the silicone hydrogel.

In one embodiment of this invention, the silicone hydrogel wound care device (which may be fabric- or polymer-reinforced and which may be a moisture barrier) encapsulates a reservoir of saline solution or hypertonic solution, which provides a source of moisture for dry wounds and optionally also delivers drugs or growth factors across the wound facing membrane, wherein the wound facing membrane and air facing membrane are either the same or different material and wherein the device is held in place by an adhesive.

The silicone hydrogel wound care device of this invention may be sealed around a wound to provide direct contact of saline, silicone hydrogel oligomers, or other fluid media to the wound bed. In this embodiment, the device may be flushed to cleanse the wound of waste products. In this embodiment, antimicrobials may be incorporated into the fluid to prevent infection and/or collagen may be added to the fluid to encourage healing and/or growth factors may be added to the fluid to encourage healing. In this embodiment, wound exudates enter the fluid media to be easily removed upon flushing and oxygen permeability is maintained by the silicone hydrogel membrane. This inventive device embodiment may be flushed either by a pair of inlet and outlet valves or through the use of a syringe and syringe septum located on the device.

Silicone hydrogel sprays in accordance with this invention may provide temporary wound dressings by polymerizing upon contact with the wound and may provide oxygen permeability and a moist wound environment. Wound dressings/sprays/liquids in accordance with this invention which employ Si—O— groups and/or other gas permeable chemicals, agents, groups, and polar groups such as ether, OH, NH—, COO—, and SO₃— allow gas permeability and moisture permeability and high water content.

Wound dressings/sprays/liquids in accordance with the present invention provide contact lens-like comfort to wounds by using hydrogels which are free of chemicals that “sting” and which are characterized by low modulus, thus avoiding biological irritation to the wounds. Wound dressings/sprays/liquids in accordance with this invention which contain hydrophilic molecules such as PVP or PEO molecules, either free or chemically bound to the bulk for the purposes of lubricating and reducing friction against tissue, may be programmed to leave the dressing and/or to modify dressing properties in order to meet the needs of different healing stages.

Other embodiments of the present invention include: silicone hydrogel dressings that contain heparin and/or other natural materials to provide optimal biocompatibility; silicone hydrogel dressings free of stimulants and adhesive groups that have minimal protein absorption/activation and have minimal ‘sting’ to the wound; silicone hydrogel dressings that have high water content and low modulus, both of which act to provide wound comfort; and silicone hydrogel dressings that have multi-layer structure with each layer designed for optimized multifunctional wound care.

### BRIEF DESCRIPTION OF THE DRAWINGS

The present invention may be more fully understood from the detailed description given below and the drawings that accompany this specification. The drawings are given by way of illustration only, and thus are not limiting of the present invention. The drawings are not necessarily to scale.

FIG. 1 is a schematic representation of a silicone hydrogel patch.

FIG. 2 is a schematic representation of a fabric-reinforced silicone hydrogel patch.

FIG. 3 is a schematic representation of a self-adhesive silicone hydrogel patch.

FIG. 4 is a schematic representation of a self-adhesive silicone hydrogel patch which is fabric reinforced.

FIG. 5 is a schematic representation of a self-adhesive silicone hydrogel patch which has a saline reservoir incorporated therein.

FIG. 6 is a schematic representation of a self-adhesive, fabric- or polymer-reinforced silicone hydrogel patch which has a saline reservoir incorporated therein.

FIG. 7 is a schematic representation of a flushable dressing.

FIG. 8 is a schematic representation of a silicone hydrogel wound care device.

FIG. 9 is a schematic representation of a temporary wound dressing provided by a silicone hydrogel spray in accordance with the present invention.

### DETAILED DESCRIPTION OF THE INVENTION

The silicone hydrogel formulation employed in the present invention may contain random and/or block copolymers or oligomers or macromers containing Si—O, C—C, Si—C, or Si—O—C bonds. The silicone copolymer is copolymerized/blended with the other polymers/monomers/macromers to obtain final formulation. The silicone hydrogel can contain crosslinking groups to obtain a complete or partially crosslinked final structure. The silicone hydrogel formulation can be preformed as films or other structures, or polymerized during application such as in the case of an adhesive formulation.

An example of silicone hydrogel formulation is composed of a copolymer of mono- or di-functional polydimethylsiloxane methacrylate, DMA, HEMA, and TRIS with or without additional crosslinking agents such as EGDMA.

Another example is composed of initiator, mono- or di-functional polydimethylsiloxane methacrylate, DMA, HEMA, and TRIS with spraying solvent. When sprayed onto wounds, polymerization will start by water, or air, or light, or heat initiating the radical polymerization.

Another example formulation is composed of mono- or di-functional polydimethylsiloxane isocyanate, polydimethylsiloxane-copolymer diisocyanate, polyethyleneglycol, Jeffamine, and catalyst. The formulation can be sprayed or applied as a paste. When applied to wounds, the isocyanate reacts with diol or diamine to form silicone polyurethaneurea hydrogel.

Another example is the foregoing isocyanate formulation plus PVP with different molecular weights ranging from 100 to 10 million. The PVP will flow to the dressing surface upon hydration to form a lubricious layer.

Another example is the foregoing isocyanate formulation plus PEO with different molecular weights ranging from 100 to 10 million. The PEO will flow to the dressing surface upon hydration to form a lubricious layer.

For wet wounds, the foregoing isocyanate-PVP formulation can be used with no or a small amount of water pre-added.

For dry wounds, the foregoing isocyanate-PVP formulation can be used with containing as much as 60% water.

The amount, structure and molecular weight PVP or PEO when added into the foregoing isocyanate formulations can be controlled to allow for a precalculated removal by washing or absorption, so that the moisture content and oxygen permeability can be programmed in the dressing to meet the needs of staged/phased wound healing.

### Additional Functionality Added to Silicone Hydrogel Materials

To improve or accelerate wound healing, the following modifications can be made to the base hydrogel material:

Antimicrobial chemical groups to remove infectious agents from the wound site. The antimicrobial groups can be covalently bound to the hydrogel material or distributed into the bulk material for slow release to the wound site. In addition, the antimicrobial agent can be linked to the polymer backbone through degradeable linkages for slow release to the wound site.

Heparin or other sulfonated polysaccharides can be covalently bound to the polymer backbone to impart antimicrobial activity. Additionally, free heparin can be released from the bulk to the wound site to help remove or inactivate infectious agents.

The base hydrogel material can be modified to allow for programmable hydrophilicity. Over the course of wound healing, it may be beneficial to change the properties of the wound dressing materials. The hydrophilicity of the hydrogel can be engineered to be programmable, e.g. the material could become less hydrophilic over the course of the wound healing.

The base hydrogel material can be modified to allow for programmable pH control. As the wound heals, it may be beneficial for the wound dressing to control the pH of the wound environment. As the wound heals, it may be beneficial to impart a slightly acidic environment to the wound site to help wound healing.

Growth factors can be added to the bulk silicone hydrogel for improved wound healing. Collagen can be added to the bulk silicone hydrogel for enhanced dermal tissue formation or angiogenesis.

Pharmaceutical agents can be added to the bulk silicone hydrogel for drug delivery.

Persons skilled in the art will be aware of silicone starting materials which can be used to make silicone hydrogels usable in implementing the present invention. Relevant disclosures may be found in the following U.S. patents, the disclosure of each of which is incorporated herein by reference: 4,686,137; 4,861,830; 4,675,361; 5,120,813; 5,235,003; 5,428,123; 5,589,563; 5,756,632; 6,692,528; 7,249,848; 7,247,692; 7,238,750; 7,201,481; 7,268,198; 6,891,010; 6,858,218; 6,849,67; 6,815,074; 5,965,631; 5,539,016; and 5,426,158.

Wounds with high exudates flow: Certain wounds can have a high release of exudates that need to be removed while preventing wound dessication. Common gauze wounds absorb wound exudates, but also can lead to wound dessication. A silicone hydrogel material can be applied to a wound with high exudates flow and the MVTR rate of the specific hydrogel material can provide controlled wound drainage.

### Examples of Wound Treatment

Wounds treatment is dependent on many factors including: dryness of the wound, stage of wound healing, degree of infection, and severity of the wound. A physician needs a “catalog” of wound care dressings or devices to address wound healing depending on the above factors. Therefore, the purpose of this disclosure is to demonstrate and list the flexibility of silicone hydrogel materials to be tailored for specific properties needed for proper wound treatment. The following list exhibits the different types of dressings or devices that can be made with specific moisture content or oxygen permeability dependent on the material composition.

Dry Wounds: It is common practice to supply moisture to dry wounds. Hydrocolloids are often used for this purpose, although they lack oxygen permeability that is also very desirable. The following are examples wound dressings made from silicone hydrogels that prevent wound desiccation while maintaining good oxygen permeability.

### Examples of Wound Dressing Applications

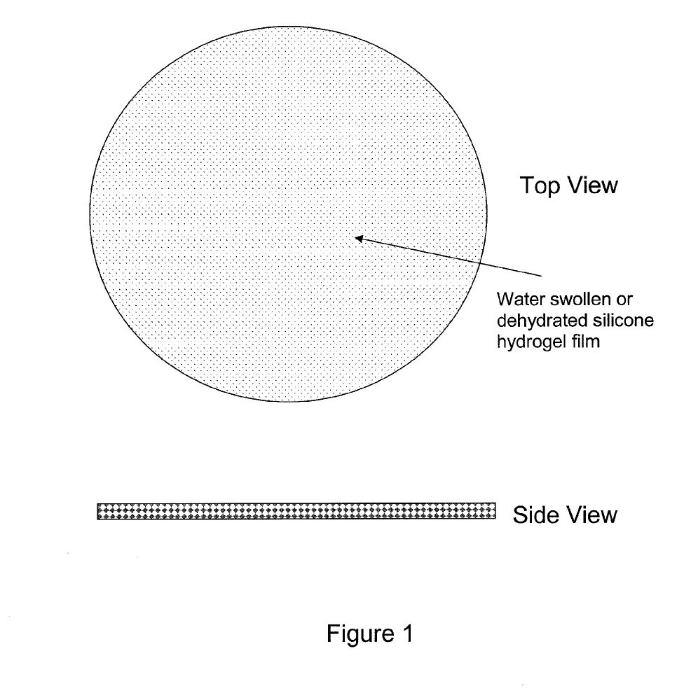

FIG. 1 illustrates a silicone hydrogel film-type wound dressing, which can initially be either hydrated or dehydrated, that is placed in contact with a wound and held in place by tape or other secondary support methods.

In a silicone hydrogel wound dressing similar to that depicted in FIG. 1, the silicone hydrogel material may be chosen to have a water content between 20 and 50%. The dressing can either be pre-hydrated or dry when applied to the wound bed. If the water content between the wound dressing and wound is higher than the surrounding atmosphere, moisture will flow out of the wound through the dressing and evaporate. If the wound healing reaches a stage where further drainage is not necessary, a silicone or silicone urethane layer can be applied to the outside of the wound dressing to prevent wound dessication.

A silicone hydrogel wound dressing similar to that depicted in FIG. 1 may be configured with programmable hydrophilicity. In some applications, it may be useful to have a material that “evolves” with the wound. Upon initial application, it may present a water content between 40 and 70%. As the wound heals, the wound dressing will slowly lose its hydrophilicity and the rate of wound drainage would proportionately decrease.

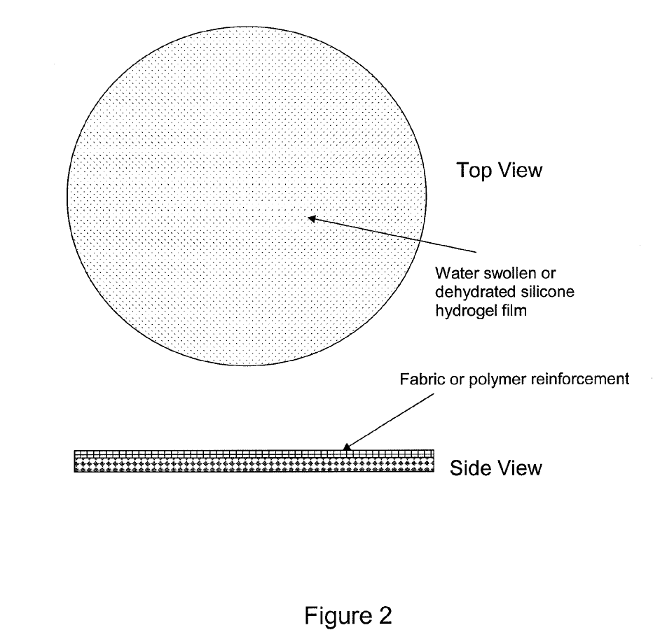

FIG. 2 illustrates a fabric-reinforced or polymer-reinforced silicone hydrogel film wound dressing, initially hydrated or dehydrated, that is placed in contact with a wound and held in place by tape or other secondary support methods. The reinforcement layer may advantageously be a moisture barrier to prevent dehydration of the silicone hydrogel.

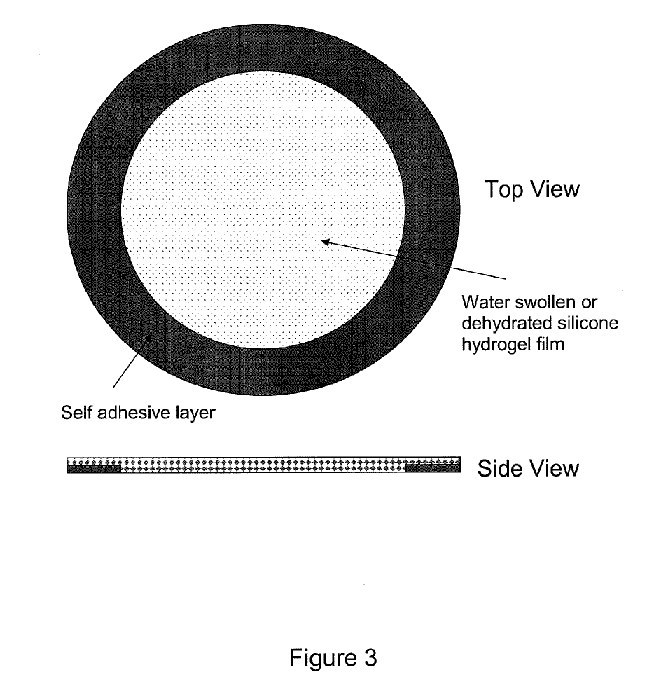

FIG. 3 illustrates a silicone hydrogel film, initially hydrated or dehydrated, that is placed in contact with a wound and is held in place by an adhesive around the wound site. The may be placed topically prior to dressing the wound, or the adhesive may be incorporated onto the film during manufacture.

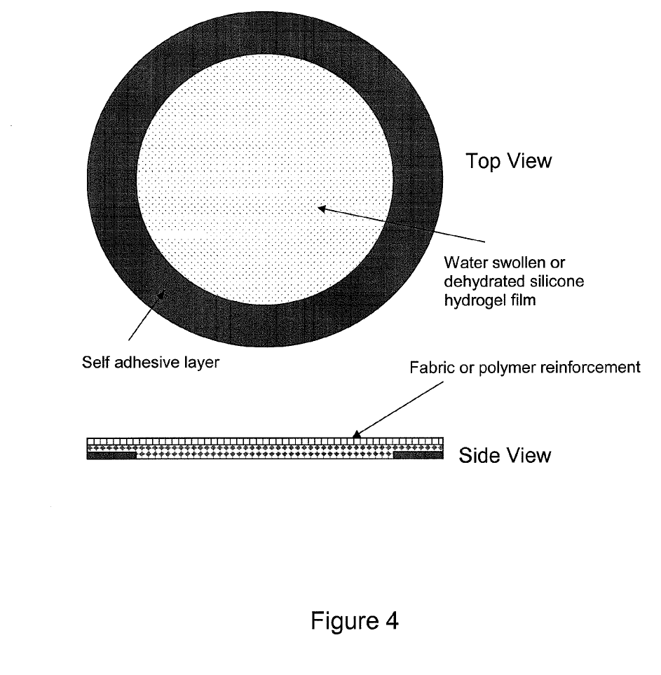

FIG. 4 illustrates a fabric- or polymer-reinforced silicone hydrogel film, which is either initially hydrated or initially dehydrated, that is placed in contact with a wound and is held in place by an adhesive around the wound site. The adhesive is either placed topically on the patient prior to dressing the wound, or is incorporated onto the film during manufacture. The reinforcement layer is advantageously formulated to be a moisture barrier to prevent dehydration of the silicone hydrogel.

In a silicone hydrogel wound dressing similar to that depicted in FIG. 4, the silicone hydrogel layer may have a water content between 50 and 70% and be prehydrated with water or saline solution. The reinforcement layer may be a silicone coated fabric or silicone urethane with low MVTR but high oxygen permeability. The silicone hydrogel will provide a moist environment for the wound, while the silicone reinforcement layer will prevent drying out of the wound.

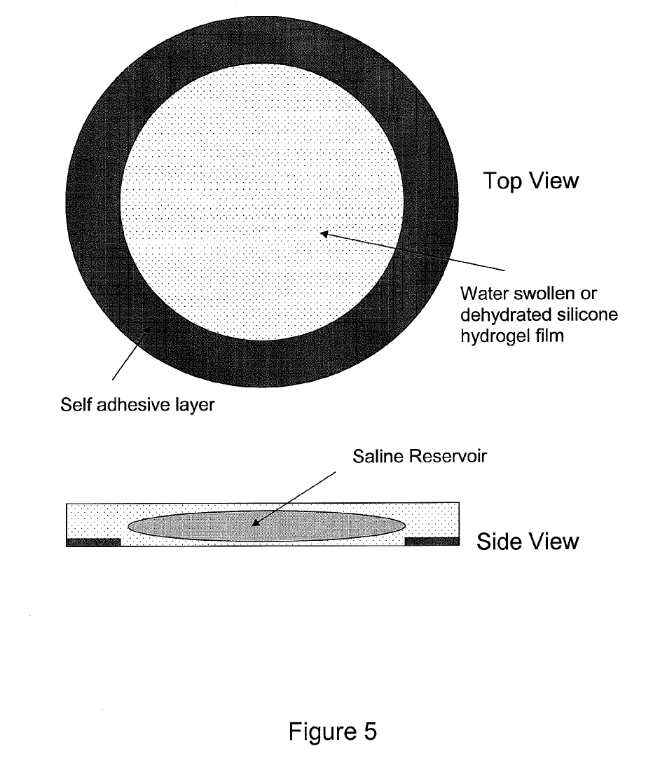

FIG. 5 illustrates a silicone hydrogel wound care device that encapsulates a reservoir of saline solution. The saline solution provides a source of moisture for dry wounds. The reservoir may also be used to deliver drugs or growth factors to the wound across the wound-facing membrane. The wound-facing membrane and the air-facing membrane may be either the same or different material. The wound dressing is held in place by an adhesive.

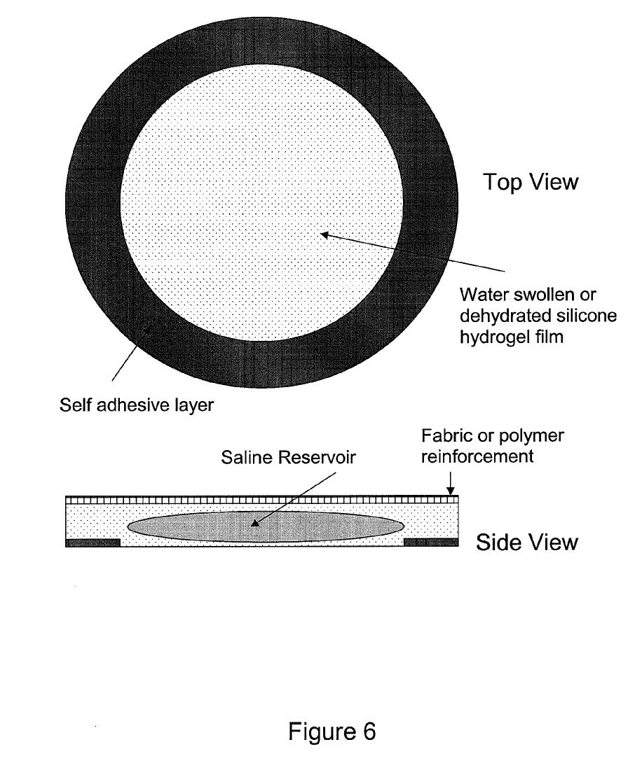

FIG. 6 incorporates a fabric- or polymer-reinforced silicone hydrogel wound care device encapsulating a reservoir of saline. The saline provides a source of moisture for dry wounds and can also be used to deliver drugs or growth factors across the wound facing membrane. The wound facing membrane and air facing membrane can be either the same or different material. The device is held in place by an adhesive. The reinforcement layer may formulated to be a moisture barrier in order to prevent dehydration of the silicone hydrogel.

In a water or saline encapsulated silicone hydrogel patch similar to that depicted in FIG. 6, the reinforcement layer may be a silicone coated fabric or silicone urethane with low MVTR but high oxygen permeability. The water or saline reservoir will provide a source of water that can transmit to the dry wound site. In addition, antimicrobial agents, growth factors, collagen, or heparin can be added to the reservoir for therapeutic purposes.

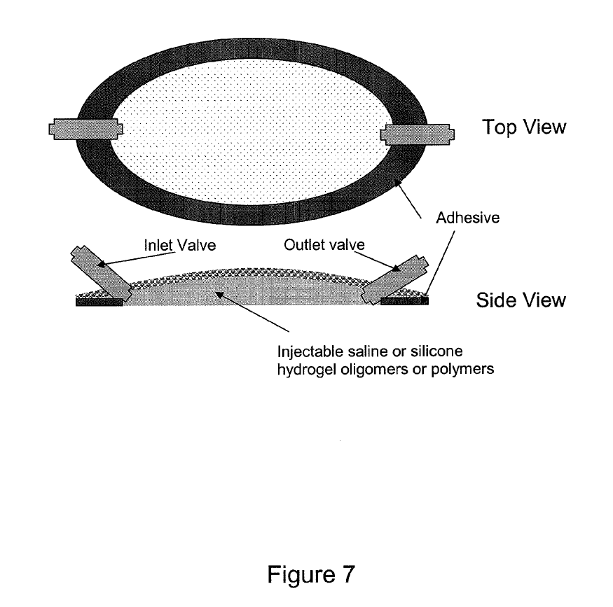

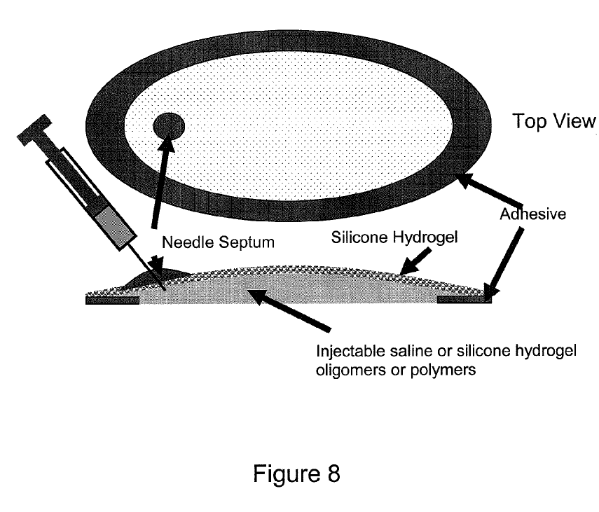

FIGS. 7 and 8 illustrate a flushable silicone hydrogel wound care device that seals around the wound and provides direct contact of saline, silicone hydrogel oligomers, or other fluid media with the wound bed. The device can be flushed to regularly cleanse the wound of waste products. Antimicrobials may be incorporated into the fluid to prevent infection. Collagen may be added to the fluid to encourage healing. Growth factors may likewise be added to the fluid to encourage healing. The wound exudates enter the fluid media so as to be easily removed upon flushing. Oxygen permeability is maintained by the silicone hydrogel membrane. The device is flushed either by a pair of inlet and outlet valves (FIG. 7), or through the use of a syringe and syringe septum located on the device (FIG. 8).

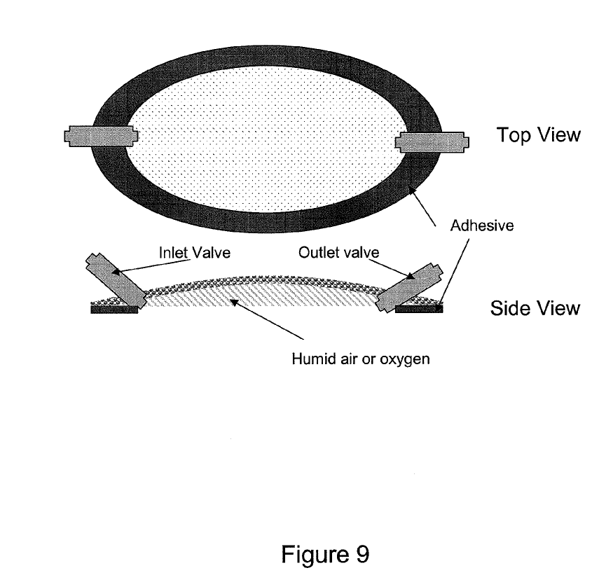

FIG. 9 illustrates a humid air or oxygen flowable silicone hydrogel wound care device which seals around the wound and provides direct contact sterile air or oxygewith the wound bed. The device can be flushed with saline to regularly cleanse the wound of waste products. Antimicrobials can be incorporated during the flushing step to prevent infection. The oxygen or air flows through the by a pair of inlet and outlet valves that also act as ports for flushing the device.

Alternatively to the pre-formed wound dressings discussed above, which are manufactured and then applied to wounds, another embodiment of the present invention provides wound dressings which are formed in place on or over the wound, typically from silicone hydrogel spray formulations. The silicone hydrogel polymerizes upon contact with the wound. The sprayed-on silicone hydrogel provides oxygen permeability and a moist wound environment. The sprayed-on wound dressing will normally degrade over time to facilitate its removal.

Wounds undergoing enzymatic debridement: When a wound is undergoing enzymatic debridement, it should remain moist and infection free. A silicone hydrogel wound dressing, as described above for dry wounds, can maintain a moist environment with high oxygen permeability and act as barrier to infection. For addition protection against infection, antimicrobial agents can be added into the device or dressing. For instance, surface active covalently bound antimicrobials such as quarternary ammonium compounds can be incorporated into the polymer formulation to remove pathogens and prevent infections. Surface active covalently bound heparin can also be attached to the dressing surface. Heparin is well known for irreversibly binding infectious agents such as bacteria, viruses, and parasites. Antimicrobial release from silicone hydrogels can also be an effective method to prevent infection. Heparin can be released into the wound site to bind with and inactivate pathogens. Other antimicrobial agents such as antibiotics or silver ions can also be released to inactivate pathogens.

Wounds undergoing the proliferative phase: During wound healing, a stage known as the proliferative phase occurs in which granulation occurs through the synthesis of collagen and production of new capillaries. The wound will then contract and epithelialization will occur. While the wound is undergoing the proliferative phase, it may be useful for the wound dressing to release bioactive agents such as collagen or growth factors.

Film dressing for wound management: A spray or liquid wound dressing formulation of silicone hydrogel is applied to the wound. The spray or liquid washes/cleans the wound and a film is left behind as a film dressing. This first film dressing can have special properties such as lubrication, containing Heparin for optimal biocompatibility, and low modulus for comfort. It can also contain free PVP or PEO that can be slowly absorbed or washed away and leaving behind a high silicone content non adhesive film for easy removal.

Multilayer dressing: After the application of the first film dressing, the same or different spray, liquid or solid dressings can be applied, forming multilayered dressing. Each layer can have different specific functions such as providing warmness, a physical barrier, a bacteria barrier, absorbent properties, etc. The multiple layer approach can also be preformed/manufactured and directly applied to wound. The modulus and elasticity of the dressing can be adjusted to comply with tissue so that the friction and stress can be minimized to reduce irritation to wound.

### Examples of Silicone Hydrogels for Tissue Adhesives/Tissue Dressing Applications

### Example 1

### Silicone Hydrogel Spray for 2^{nd} and 3^{rd} Degree Burns

A mixture of silicone hydrogel spray is composed of an initiator, difunctional PDMS methacrylate, DMA, HEMA, and TRIS with a spraying solvent.

| Ingredient | Weight % |
|---|---|
| Difunctional PDMS methacrylate | 25 |
| DMA | 15 |
| HEMA | 15 |
| TRIS | 20 |
| Alcohol/water | 24.9 |
| UV/Visible light initiator | 0.1 |

The hydrogel is sprayed onto a burn and allowed to polymerize over the wound under UV/visible light. Once polymerized, the hydrogel acts as an artificial skin and will provide a high moisture environment, oxygen permeability, and a barrier to infection. This method of wound care is superior to current bandages or wound dressings because it will provide an effective microbial barrier, prevent wound desiccation, and allow high oxygen permeability for tissue healing. The high oxygen permeability and moisture content will also help prevent scarring.

### Example 2

### Silicone Hydrogel Spray for 2^{nd} and 3^{rd} Degree Burns

A mixture of silicone hydrogel emulsion spray is composed of an initiator, difunctional PDMS methacrylate, DMA, and TRIS.

| Ingredient | Weight % |
|---|---|
| Difunctional PDMS methacrylate | 25 |
| DMA | 30 |
| TRIS | 20 |
| Water | 24.9 |
| UV/Visible light initiator | 0.1 |

The hydrogel emulsion is sprayed onto a burn and allowed to polymerize over the wound under UV/visible light. Once polymerized, the hydrogel acts as an artificial skin and will provide a high moisture environment, oxygen permeability, and a barrier to infection. This method of wound care is superior to current bandages or wound dressings because it will provide an effective microbial barrier, prevent wound desiccation, and allow high oxygen permeability for tissue healing. The high oxygen permeability and moisture content will also help prevent scarring.

### Example 3

### Silicone Hydrogel Spray for 2^{nd} and 3^{rd} Degree Burns

A mixture of silicone hydrogel emulsion spray is composed of an initiator, difunctional PDMS methacrylate, DMA, PVP and TRIS.

| Ingredient | Weight % |
|---|---|
| Difunctional PDMS methacrylate | 20 |
| DMA | 15 |
| PVP K-90 | 25 |
| TRIS | 15 |
| Water | 24.9 |
| UV/Visible light initiator | 0.1 |

The hydrogel emulsion is sprayed onto a burn and allowed to polymerize over the wound under UV/visible light. Once polymerized, the hydrogel acts as an artificial skin and will provide a high moisture environment, oxygen permeability, and a barrier to infection. This method of wound care is superior to current bandages or wound dressings because it will provide an effective microbial barrier, prevent wound desiccation, and allow high oxygen permeability for tissue healing. The high oxygen permeability and moisture content will also help prevent scarring. The PVP molecules will be absorbed or washed away slowly to give a gradually increasing hydrophobic environment that provides a decreasing moisture permeability to promote healing at different wound healing stages.

### Example 4

### Silicone Hydrogel Patch for External Dermal Sutures

A film of silicone hydrogel is made by reacting di-functional PDMS methacrylate, DMA, HEMA, DMEA catalyst and Tris while exposed to UV radiation.

| Composition | Weight % |
|---|---|
| Difunctional PDMS methacrylate | 30 |
| DMA | 20 |
| HEMA | 20 |
| TRIS | 30 |

The film is then cut to the appropriate size and adhered around the outside of the wound using an approved cyanoacrylate tissue adhesive. The hydrogel film would provide a high oxygen environment for tissue healing and prevent wound dessication.

### Example 5

### Silicone Hydrogel Patch for External Dermal Sutures

A film of silicone hydrogel is made by reacting di-functional PDMS methacrylate, DMA, HEMA, DMEA catalyst and Tris while exposed to UV radiation.

| Composition | Weight % |
|---|---|
| Difunctional PDMS methacrylate | 25 |
| DMA | 15 |
| PVP K-90 | 20 |
| HEMA | 15 |
| TRIS | 25 |

The film is then cut to the appropriate size and adhered around the outside of the wound using an approved cyanoacrylate tissue adhesive. The hydrogel film would provide a high oxygen environment for tissue healing and prevent wound dessication. The PVP molecules will be absorbed or washed away slowly to give a gradually increasing hydrophobic environment that provides a decreasing moisture permeability to promote healing at different wound healing stages.

### Example 6

### Silicone Hydrogel Patch with Hypotonic Solution Reservoir for Wound Drainage

A Silicone hydrogel film is made reacting di-functional PDMS methacrylate, DMA, HEMA, DMEA catalyst and Tris while exposed to UV radiation.

| Composition | Weight % |
|---|---|
| Difunctional PDMS methacrylate | 20 |
| DMA | 30 |
| HEMA | 30 |
| TRIS | 20 |

A silicone polyurethane film (Pursil 35 80A or Carbosil 60 80A) is then placed on top of the Si-hydrogel and sealed around the outside creating a sandwich structure to create a water barrier to the outside environment.

The inside of the sandwich structure is then filled with a hypotonic solution. The patch is then adhered to the outside of the wound using an approved cyanoacrylate. Exudate from the wound is allowed to permeate through the si-hydrogel membrane due to the hypotonic nature of the reservoir solution. The wound moisture content is maintained by the passage of water molecules from the solution reservoir to the wound. As needed, the solution reservoir can be drained and refilled with fresh hypotonic solution. This will allow proper wound drainage while prevent exposure or re-exposure to pathogens. Such a wound care patch is superior to current bandages or tissue dressings due to the prevention of wound dessication and re-exposure to environments in which pathogens could be present.

### Example 7

A dense semipermeable membrane is made from PurSpan C, a polyurea urethane with polycarbonate and silicone co-softsegments. PurSpan C has silicone hydrogel like properties such as being water soluble, provides oxygen permeation, and can allow exudates to permeate through the film. A silicone PurSil 35 80A film is then placed on top of the Si-hydrogel and sealed around the outside creating a sandwich structure to create a water barrier to the outside environment. The inside of the sandwich structure is then filled with a hypotonic solution. The patch is then adhered to the outside of the wound using an approved cyanoacrylate. Exudate from the wound is allowed to permeate through the PurSpan C membrane due to the hypotonic nature of the reservoir solution. The wound moisture content is maintained by the passage of water molecules from the solution reservoir to the wound. As needed, the solution reservoir can be drained and refilled with fresh hypotonic solution. This will allow proper wound drainage while prevent exposure or re-exposure to pathogens. Such a wound care patch is superior to current bandages or tissue dressings due to the prevention of wound desiccation and re-exposure to environments in which pathogens could be present.

Persons skilled in the art will readily recognize that additional variations of the above-described implementations may be reached without departing from the spirit and scope of the present invention.

1.-31. (canceled)

32. A wound dressing comprising a silicone hydrogel formed as a film which has both oxygen permeability and moisture-absorptivity, wherein said silicone hydrogel is a reaction product of a polymerizable silicone and a hydrophilic crosslinking agent, wherein said oxygen permeability is contributed by the hydrophobic silicone moiety and said moisture-absorptivity is provided by said hydrophilic components.

33. The oxygen-permeable, moisture absorptive wound dressing of claim 32, wherein said polymerizable silicone is a polydimethylsiloxane methacrylate and said hydrophilic crosslinking agent is at least one member selected from the group consisting of N,N-dimethylacrylamide (DMA), 2-hydroxyethyl methacrylate (HEMA), and trimethylsiloxy silane (TRIS).

34. The oxygen-permeable, moisture absorptive wound dressing of claim 33, wherein said silicone hydrogel formulation is composed of a copolymer of mono- or difunctional polydimethylsiloxane methacrylate, DMA, HEMA, and TRIS, and optionally an additional crosslinking agent.

35. The oxygen-permeable, moisture absorptive wound dressing of claim 32, which is pre-formed as a film from a composition comprising 15-35 weight-% difunctional PDMS methacrylate, 10-35 weight-% DMA, 10-35 weight-% HEMA, and 15-30 weight-% TRIS.

36. The oxygen-permeable, moisture absorptive wound dressing of claim 33, which contains as an additional crosslinking agent ethyleneglycol dimethacrylate (EGDMA).

37. The oxygen-permeable, moisture absorptive wound dressing of claim 32, which is formed from a spray of a composition which comprises 15-30 weight-% difunctional PDMS methacrylate, 10-35 weight-% DMA, up to 20 weight-% HEMA, 10-25 weight-% TRIS₅ and 15-35 weight-% water,

38. The oxygen-permeable, moisture absorptive wound dressing of claim 32, comprising initiator, mono- or di-functional polydimethylsiloxane methacrylate, DMA, HEMA, and TRIS, with spraying solvent, configured so that when the dressing is sprayed onto a wound, polymerization is started by water or air or light or by heat initiation of the radical polymerization.

39. The oxygen-permeable, moisture absorptive wound dressing of claim 32, comprising mono- or di-functional polydimethylsiloxane isocyanate, polydimethylsiloxane-copolymer diisocyanate, polyethyleneglycol, Jeffamine, and catalyst, formulated so that it can be sprayed onto a wound or applied to the wound as a paste, wherein upon application to a wound, the isocyanate reacts with diol or diamine to form silicone polyurethaneurea hydrogel.

40. The oxygen-permeable, moisture absorptive wound dressing of claim 39, wherein the formulation further contains PVP with a molecular weight in the range 100 to 10 million, wherein the PVP flows to the dressing surface upon hydration to form a lubricious layer.

41. The oxygen-permeable, moisture absorptive wound dressing of claim 32, formulated with no water or up to 5% water pre-added for use with wet wounds.

42. The oxygen-permeable, moisture absorptive wound dressing of claim 32, formulated with from 40% to 60% water pre-added for use with dry wounds.

43. A silicone hydrogel film-type oxygen-permeable, moisture absorptive wound dressing in accordance with claim 32, placed in contact with a wound and held in place by tape or other secondary support methods.

44. A fabric- or polymer-reinforced silicone hydrogel film-type oxygen-permeable, moisture absorptive wound dressing in accordance with claim 32, placed in contact with a wound and held in place by tape or other secondary support methods, wherein the reinforcement layer is optionally a moisture barrier to prevent dehydration of the silicone hydrogel.

45. A silicone hydrogel film-type The oxygen-permeable, moisture absorptive wound dressing in accordance with claim 32, placed in contact with a wound and is held in place by an adhesive around the wound site, wherein the adhesive is either placed topically prior to dressing the wound, or is incorporated into the film during manufacture.

46. A fabric- or polymer-reinforced silicone hydrogel film-type oxygen-permeable, moisture absorptive wound dressing in accordance with claim 32, placed in contact with a wound and is held in place by an adhesive around the wound site, wherein the adhesive is either placed topically prior to dressing the wound, or is incorporated into the film during manufacture, and wherein the reinforcement layer is optionally a moisture barrier to prevent dehydration of the silicone hydrogel.

47. A silicone hydrogel oxygen-permeable, moisture absorptive wound care device according to claim 32, wherein said device encapsulates a reservoir of saline solution which provides a source of moisture for dry wounds and optionally also delivers drugs or growth factors across the wound facing membrane, wherein the wound facing membrane and air facing membrane are either the same or different material and wherein the device is held in place by an adhesive.

48. A fabric- or polymer-reinforced silicone hydrogel oxygen-permeable, moisture absorptive wound care device encapsulating a reservoir of saline according to claim 47, wherein the reinforcement layer is a moisture barrier to prevent dehydration of the silicone hydrogel.

49. A silicone hydrogel oxygen-permeable, moisture absorptive wound care device according to claim 32, wherein said device encapsulates of hypotonic solution which provides a source of moisture for dry wounds and optionally also delivers drugs or growth factors across the wound facing membrane, wherein the wound facing membrane and air facing membrane are either the same or different material and wherein the device is held in place by an adhesive.

50. A fabric- or polymer-reinforced silicone hydrogel oxygen-permeable, moisture absorptive wound care device encapsulating a reservoir of hypotonic solution according to claim 49, wherein the reinforcement layer is a moisture barrier to prevent dehydration of the silicone hydrogel.

51. A silicone hydrogel oxygen-permeable, moisture absorptive wound care device in accordance with claim 32 which seals around a wound and provides direct contact of saline, silicone hydrogel oligomers, or other fluid media to the wound bed, wherein said device is adapted be flushed to cleanse the wound of waste products.

52. The flushable silicone hydrogel oxygen-permeable, moisture absorptive wound care device of claim 51, wherein antimicrobials are incorporated into the fluid to prevent infection and/or collagen is added to the fluid to encourage healing and/or growth factors are added to the fluid to encourage healing, and wherein the wound exudates enter the fluid media to be easily removed upon flushing and oxygen permeability is maintained by the silicone hydrogel membrane and the device is flushed either by a pair of inlet and outlet valves or through the use of a syringe and syringe septum located on the device.

53. A humid air or oxygen flowable silicone hydrogel oxygen-permeable, moisture absorptive wound care device in accordance with claim 32, which seals around a wound and provides direct contact sterile air or oxygen to the wound bed, which device can be flushed with saline to cleanse the wound of waste products, wherein antimicrobials can be incorporated during the flushing step to prevent infection, and wherein oxygen or air flows through the device by way of a pair of inlet and outlet valves that also act as ports for flushing the device.

54. A silicone hydrogel spray in accordance with claim 32, which provides a temporary wound dressing by polymerizing upon contact with the wound and which provides oxygen permeability and a moist wound environment.
